(19) United States
(12) Patent Application Publication
(10) Pub. No.: US 2002/0019012 A1
(43) Pub. Date: Feb. 14, 2002

(54) Method and apparatus for predicting a signal peptide cleavage site

(76) Inventor: Kuo-Chen Chou, Kalamazoo, MI (US)

Correspondence Address: MARSHALL, O'TOOLE, GERSTEIN, MURRAY & BORUN, 6300 SEARS TOWER, 233 SOUTH WACKER DRIVE, CHICAGO, IL, 60606-6402, US

(21) Appl. No.: 09/837,989

(22) Filed: Apr. 19, 2001

Related U.S. Application Data
(60) Provisional application No. 60/198,596, filed on Apr. 19, 2000.

(51) Int. Cl. G01N 33/53; G06F 19/00; G01N 33/48; G01N 33/50

(52) U.S. Cl. 435/7.1; 702/19

(57) ABSTRACT

A method and apparatus for predicting a signal peptide cleavage site associated with an amino acid sequence is provided. The system determines a size (X+Y) for a scanning window based on a positive training data set and a negative training data set. The scanning window has a signal peptide portion of length X and a mature protein portion of length Y. The training data set is indicative of a plurality of amino acid sequences with known peptide cleavage sites. The method then scans the window across the amino acids from an amino acid sequence suspected of containing a signal peptide looking for the most likely cleavage site based on the training data.

### RELATED APPLICATION

This application claims priority from provisional application serial No. 60/198,596, filed Apr. 19, 2000.

### TECHNICAL FIELD OF THE INVENTION

The present invention relates in general to a method and apparatus for characterizing proteins and in particular to predicting a signal peptide cleavage site associated with an amino acid sequence and applications therefor.

### BACKGROUND OF THE INVENTION

Protein signal sequences, also called topogenic signals or signal peptides, play a central role in the targeting and translocation of nearly all secreted proteins and many integral membrane proteins in both prokaryotes and eukaryotes. The signal peptides from various proteins generally consist of three structurally, and possibly functionally distinct, regions: (1) an amino terminal (N-terminal) positively charged n-region, (2) a central hydrophobic h-region, and (3) a neutral but polar carboxy terminal (c-region). The determination of protein signal sequences is an important tool for pharmaceutical scientists who genetically modify bacteria, plants, and animals to produce effective drugs (especially therapeutic proteins) and bioinformaticists who analyze sequence information to discern and predict properties of newly discovered molecules. By adding a specific tag to a desired protein, a scientist is able to select the protein for excretion. In this manner, the protein is easier to harvest. For example, scientists may wish to express a protein as a fusion protein comprising a preferred N-terminal sequence fused to a mature sequence of a desired protein.

However, to effectively use this technique, the signal peptides must be identified. Since the number of protein sequences entered into data banks is rapidly increasing, it is time-consuming and expensive to identify the signal peptides using traditional laboratory experiments involving expression, purification, and characterization of mature proteins. The number of sequence entries in SWISS-PROT in 1987 was 1,266. In 1988 the number increased to 3,497, and in 1997 it was up to 10,092. The growth of GenBank and other sequence databases also has been phenomenal.

Most of the existing methods for predicting signal peptides from sequence information are based on neutral networks. However, the computational cost associated with training the neural networks is high and the prediction accuracy is often lower than the traditional analytical methods.

For all of the reasons, it is highly desirable to develop a fast, accurate, and inexpensive computer algorithm to identify signal peptides and predict their cleavage sites based on sequence information alone, such as deduced amino acid sequence derived from polynucleotide sequences.

### SUMMARY OF THE INVENTION

In one aspect, the invention is directed to a method of identifying signal peptides and predicting their cleavage sites. The method determines a size (X+Y) for a scanning window based on a training data set. Preferably, the scanning window has a signal peptide portion of length X and a mature protein portion of length Y. The training data set is indicative of a plurality of amino acid sequences with known peptide cleavage sites. Preferably, the training data includes a positive set and a negative set. The method receives a first data set representing (X+Y) amino acids from an amino acid sequence suspected of containing a signal peptide, and determines a first probability associated with the first data set based on the training data set. Subsequently, the method receives a second data set representing (X+Y) amino acids from the same amino acid sequence (e.g., the window is moved one position), and determines a second probability associated with the second data set. The data set with the higher probability is chosen, thereby predicting the cleavage site to be located between X and Y.

In another aspect, the invention is directed to an apparatus for predicting a signal peptide cleavage site associated with an amino acid sequence. The apparatus includes a memory device which stores a software program and a central processing unit operatively coupled to the memory device. The central processing unit executes the software program. The software program determines a size (X+Y) for a scanning window based on a training data set. Preferably, the scanning window has a signal peptide portion of length X and a mature protein portion of length Y. The training data set is indicative of a plurality of amino acid sequences with known peptide cleavage sites. The software program receives a first data set representing (X+Y) amino acids from an amino acid sequence suspected of containing a signal peptide, and determines a first probability associated with the first data set based on the training data set. Subsequently, the software program receives a second data set representing (X+Y) amino acids from the same amino acid sequence, and determines a second probability associated with the second data set. The data set with the higher probability is chosen, thereby predicting the cleavage site to be located between X and Y.

In yet another aspect, the invention is directed to a method for the preparation of a chimeric polynucleotide comprising an expression control sequence which encodes for a signal peptide, fused in frame with a nucleotide sequence which encodes for a mature peptide sequence, the software program representing the step of determining a signal peptide cleavage site associated with the expression control sequence. Exemplary methods and compositions for recombinant protein production using the signal peptide/native protein cleavage site based technologies described herein are described in further detail below.

An “expression control sequence” is here defined minimally as a polynucleotide encoding for methionine and serving as a site for initiation of translation in a prokaryotic or eukaryotic host cell. Preferably, the expression control sequence also includes any of the following:

a eukaryotic signal peptide that includes a methionine and additional residues that will be recognized by a selected host cell to direct secretion of a mature peptide attached thereto;

upstream promoters and enhancers;

an initiator methionine and upstream fusion partner that can be cleaved as desired after expression of the polynucleotide;

or a tag sequence;

with the provision that the chimeric polynucleotide does not comprise the original signal peptide sequence of the protein fused to and immediately upstream of the predicted mature protein portion of the polypeptide. Such expression control sequences include polynucleotides encoding for methionine, methionine-lysine initiator sequences, an initiator methionine coupled with a GST-fusion partner, or methionine coupled with a poly-histidine sequence. One preferred class of expression control sequences comprises sequences that encode heterologous signal peptides (i.e., signal peptides found on other proteins and artificial signal peptides). Such a list is not intended as a limitation upon the polynucleotides which may be used, but as an example of possible polynucleotide constructs which are embraced by the invention.

Several methods of preparing polynucleotides which encode for a known amino acid residue sequence have been developed, and can be found, e.g., in Ausubel, et al. (Eds.), Protocols in Molecular Biology, John Wiley & Sons (1994-99) or Sambrook et al. (Eds.), Molecular Cloning: A Laboratory Manual, Cold Spring Harbor Press, Cold Spring Harbor, N.Y. (1989), herein incorporated by reference. Other methods comprising modifications of the above referenced techniques will be obvious to those skilled in the art. Such techniques may make use of the “redundancy” in the genetic code. For example, various codon substitutions, such as the silent changes which produce various restriction sites, may be introduced to optimize expression in a particular prokaryotic or eukaryotic system. Upon preparation of the chimeric polynucleotide sequence, a host cell may be transformed or transfected with the sequence, and the host cell grown under conditions which permit the expression of a recombinant polypeptide encoded by the chimeric nucleotide sequence. The term “recombinant,” when used herein to refer to a polypeptide or protein, means that a polypeptide or protein is derived from recombinant (e.g., microbial or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells. Preferably, the host cell is a eukaryotic cell that recognizes and cleaves the signal peptide and secretes the resultant mature polypeptide encoded by the chimeric polynucleotide. The resulting expressed polypeptide can then be purified from the host cell or the growth medium of the cell using several methods, e.g., SDS-PAGE, affinity chromatography, or ion-exchange chromatography. Many protein purification techniques are available, and are well-known to those skilled in the art. Alternatively, the host cell may cleave the signal peptide portion of the polypeptide and secrete the mature protein sequence, which may then be purified as described above.

In another aspect, the invention is directed to a method for the recombinant production of a polypeptide using chimeric polynucleotides as described above, the software program of the invention representing the step of determining the likely point of cleavage between the signal peptide and the mature protein. Thus, the invention provides a method that involves predicting a signal peptide sequence as described in detail herein, and that further comprises a step of preparing a chimeric nucleotide sequence comprising an expression control nucleotide sequence fused in frame with a nucleotide sequence encoding the mature protein portion of the amino acid sequence. In a preferred embodiment, the method further includes steps of transforming or transfecting a host cell with the chimeric nucleotide sequence; and growing the host cell under conditions to permit expression of the polypeptide encoded by the chimeric nucleotide sequence. In a highly preferred embodiment, the method further comprises a step of purifying the polypeptide from the host cell or the growth media of the cell. Where the expression control sequence includes a heterologous signal peptide sequence fused in frame with the nucleotide sequence encoding the mature protein, and where the host cell is a eukaryotic cell that recognizes and cleaves the heterologous signal peptide and secretes a polypeptide encoded by the chimeric nucleotide sequence and lacking the signal peptide, it is possible to purify the mature protein portion of the chimeric polypeptide from the growth medium of the cell.

In still another aspect, the invention is directed to a method for the preparation of a synthetic polypeptide comprising a predicted mature protein portion of a polypeptide and lacking a predicted signal peptide portion, the software program of the invention representing the step of determining the predicted point of cleavage. “Synthetic”, when used herein to refer to a polypeptide or protein, refers to a polypeptide or protein made through non-biological (e.g., chemically synthesized without the use of cellular machinery) processes. Such synthetic peptides may be prepared by any of several methods, e.g., solid phase peptide synthesis. Further methods can be found in Merrifield et al., J. Am. Chem. Soc., 85:2149 (1963); Houghten et al., Proc Natl Acad. Sci. USA, 82:5132 (1985); and Stewart and Young, Solid Phase Peptide Synthesis, Pierce Chemical Co., Rockford, Ill. (1984), herein incorporated by reference. Such techniques may further be automated by addition of a peptide synthesizer, which can be programmed to repeatedly perform the addition steps to produce a peptide constituting a given amino acid sequence. Upon preparation of the polypeptide, it may be purified using any of the methods (e.g., SDS-PAGE, affinity chromatography, or ion-exchange chromatography) described above.

In yet another aspect, the invention is directed to a computer readable medium storing a software program, the software program representing the step of predicting a signal peptide cleavage site associated with an amino acid sequence. The software program representing a step of determining a size (X+Y) for a scanning window based on a training data set. Preferably, the scanning window has a signal peptide portion of length X and a mature protein portion of length Y. The training data set is indicative of a plurality of amino acid sequences with known peptide cleavage sites. The software program also represents a step of receiving a first data set representing (X+Y) amino acids from an amino acid sequence suspected of containing a signal peptide, and a step of determining a first probability associated with the first data set based on the training data set. A subsequent step represents receiving a second data set representing (X+Y) amino acids from the same amino acid sequence, and a step of determining a second probability associated with the second data set. The data set with the higher probability is chosen by the software program represented, thereby predicting the cleavage site to be located between X and Y.

### BRIEF DESCRIPTION OF THE DRAWINGS

These and other features and advantages of the present invention will be apparent to those of ordinary skill in the art in view of the detailed description of the preferred embodiment which is made with reference to the drawings, a brief description of which is provided below.

FIG. 1a is a symbolic representation of an amino acid sequence.

FIG. 1b is a symbolic representation of an amino acid sequence with a sliding window.

FIG. 1c is a histogram showing the frequencies of 20 native amino acids occurring at the subsites proximal to the cleavage site.

FIG. 2 is a block diagram of a computing device capable of executing some or all of the method of the present invention.

FIGS. 3a-3c is a flowchart illustrating a method of predicting a signal peptide cleavage site associated with an amino acid sequence.

FIG. 4 is a flowchart illustrating another method of predicting a signal peptide cleavage site associated with an amino acid sequence.

### DETAILED DESCRIPTION OF THE PREFERRED EMBODIMENTS

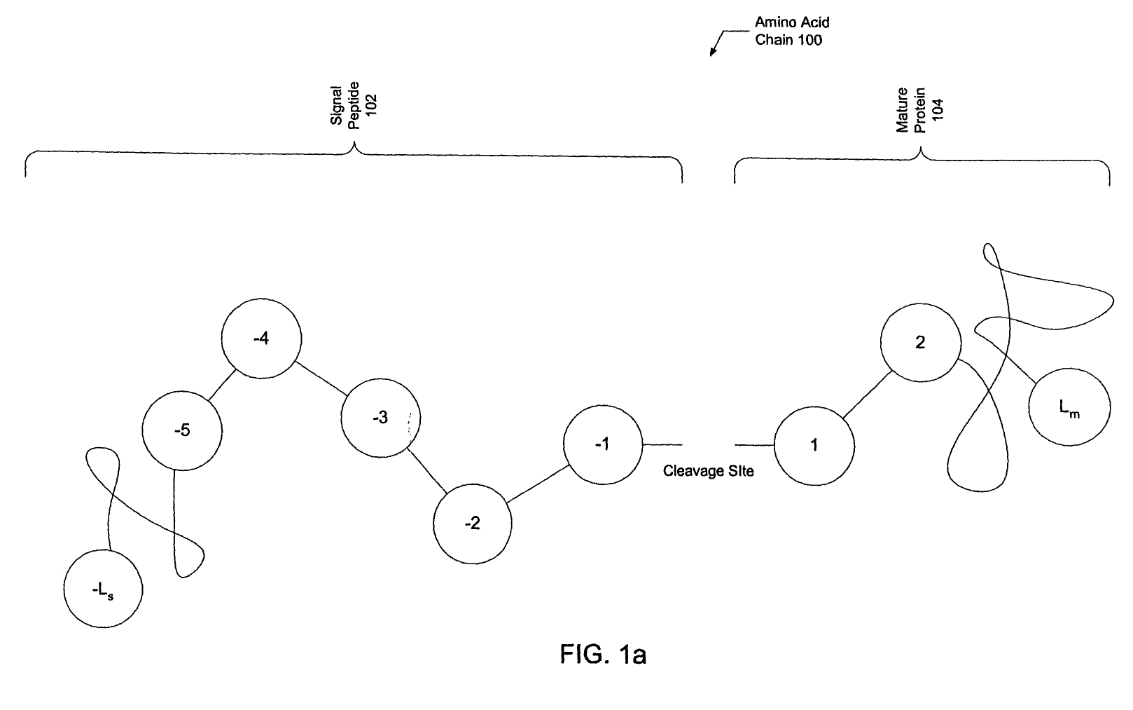

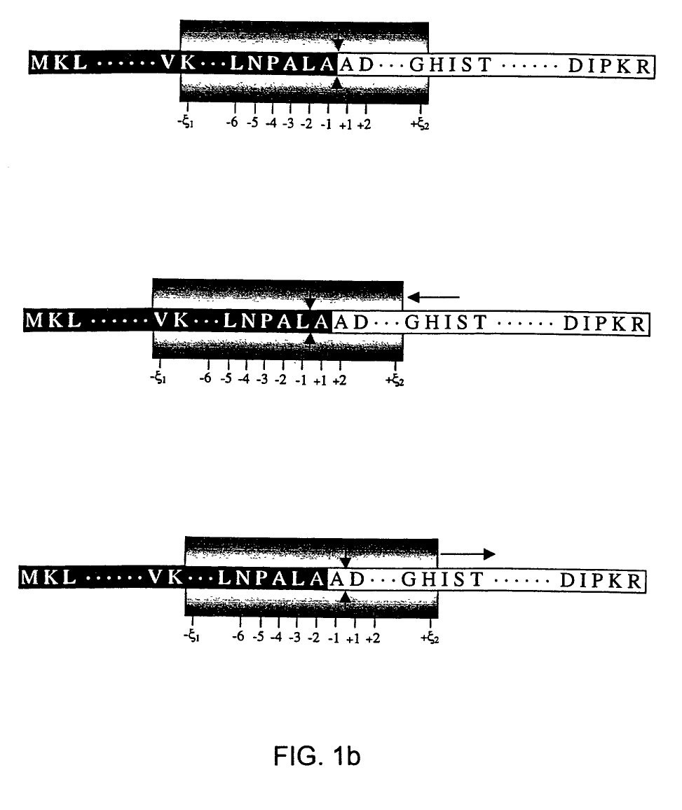

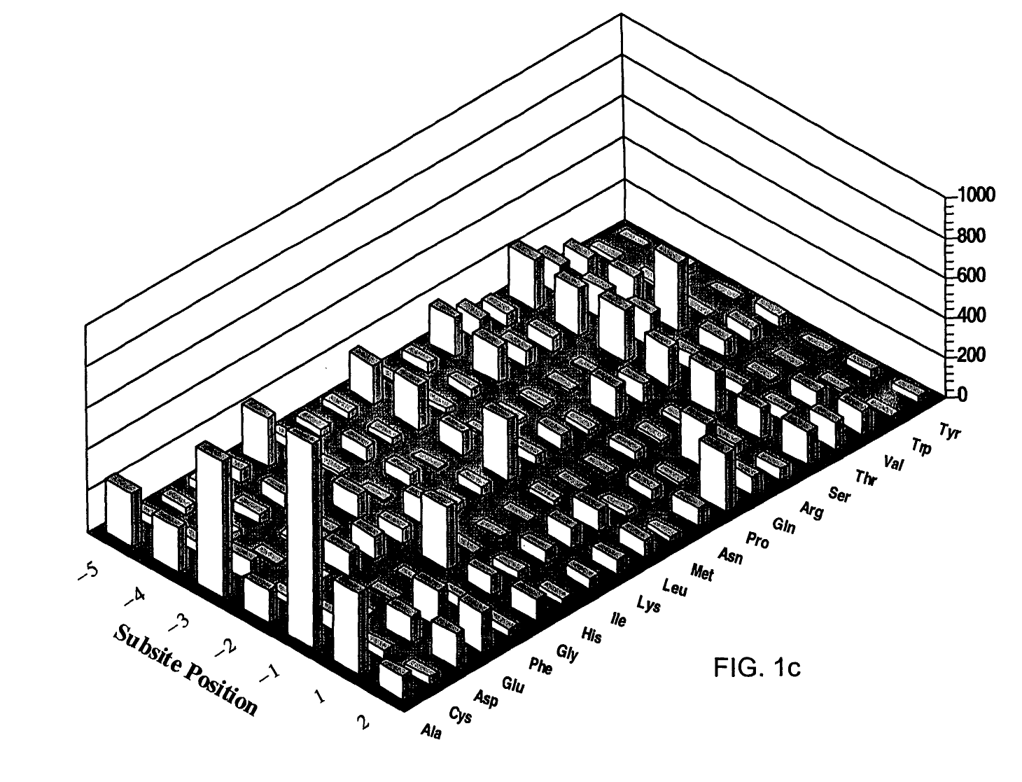

A symbolic representation of an amino acid chain 100 is illustrated in FIG. 1a. The amino acid 100 includes a signal peptide portion 102 and a mature protein portion 104. The signal peptide portion 102 may be cleaved off while the mature protein portion 104 is translocated through the membrane of a cell. The length of the signal peptide 102 varies from protein to protein. Typically, the shortest signal peptides 102 are only eight amino acids long (Ls=8), and the longest signal peptide 102 may be as long as ninety amino acids (Ls=90). However, signal peptides 102 are usually between 18 and 25 amino acids long.

In order to determine where the signal peptide portion 102 ends and the mature protein portion 104 begins, the amino acid chain 100 may be statistically characterized by a sequence symbolized as [−L1, +L2]. L1 represents a number of amino acid residues which belong to the signal peptide portion 102. L2 represents a number of residues which belong to the mature protein portion 104. The cleavage site is located between residues −1 and +1. The [−L1, +L2] sequence serves as a window to search for the secretion-cleavable site along the amino acid chain 100 and determine the transition from the signal peptide 102 to the mature protein 104 (see FIG. 1b). For example, if L1=6 and L2=2, the window is [−6, +2]. Of course, a person of ordinary skill in the art will readily appreciate that the method described herein may be used to cover any values of L1 and L2 without departing from the scope and spirit of the present invention.

This example sequence can generally be expressed as R−6R−5R−4R−3R−2R−1R+1R+2, where R−6 represents the amino acid residue at the nascent protein sequence position −6, R−5 the residue at the position −5, etc. The site at location (−1, +1), (i.e., the location between R−1 and R+1 of the sequence) is the cleavage site during the secretion process. All residues ahead of this site in the nascent protein constitute the signal peptide portion 102, and all residues after this site constitute the mature protein portion 104.

The attributes of the secretion-cleavable set and non-secretion-cleavable set may be expressed as Ψ0+ and Ψ0− respectively. Ψ0+(R−6R−5R−4R−3R−2R−1R+1 R+2)=P+−6(R−6)P+−5(R−5)P+−4(R−4)P+−3(R−3)P+−2(R2)P+−1 (R−1 )P++1(R+1)P++2(R+2) and Ψ0−(R−6R−5R−4R−3R−2R−1 R+1 R+2)=P−−6(R−6)P−−5(R−5)P−−4(R−4)P−−3(R−3)P−−2(R−2)P−−1(R−1)P−+1(R+1)P−+2(R+2), where Pi (Ri) is the probability of amino acid Ri occurring at the subsite i (=−6, −5, . . . , −1, +1, +2) for the sequences with a secretion-cleaved site at (−, +1), and P− (Ri) is the corresponding probability for the sequences without any secretion-cleaved site or for those with a secretion-cleaved site located at a position other than (−1, +1). The values of the former can be derived from a positive training data set S0+ consisting of only those sequences which have a secretion-cleaved site between R-1 and R+1, and the values of the latter can be derived from a negative training data set S0− consisting of only those sequences which have no secretion-cleaved site at all or have one but its location is at any position but (−1, +1).

The subscript 0 of Ψ indicates that the attribute function is formed by independent probabilities in which no coupling effect between subsites is included. However, in reality the protein subsites are often coupled with one another. For example, analysis of certain data indicates that the amino acid residues at the subsites −3, −1, and +1 are frequently occupied by Ala. A histogram showing the frequencies of 20 native amino acids occurring at the subsites proximal to the cleavage site is illustrated in FIG. 1c. As shown, the frequency of Ala at subsites −3, −1, and +1 is overwhelming in comparison with the other 19 amino acids.

This finding, in combination with the fact that these sites (−3, −1, and +1) are near the cleavage site, suggests that a highly special match between the signal peptidase and the secretory protein at these subsites is required during the cleaving process. Accordingly, a method for predicting signal peptides may take the coupling among these three key subsites into account using conditional probability. Of course, a person of ordinary skill in the art will readily appreciate that any subsites may be used. For example, sites (−2, −1, +1), (−3, −1, +1), or (−3, −2, −1, +1) may be used.

Using this method, the attributes of the secretion-cleavable set and non-secretion-cleavable set may be expressed as Ψ+ and Ψ− respectively. Ψ+(R−6R−5R−4R−3R−2R−1 R+1 R+2)=P+−6(R−6)P+−5(R−5)P+−4(R−4)P+−3(R−3)P+−2(R−2)P+−1(R−1|R−3)P++1 (R+1|R−1)P++2(R+2) and Ψ−(R−6R−5R−4R−3R−2R−1 R+1 R+2)=P−−6(R−6)P−−5(R−5)P−−4(R−4)P−−3(R−3)P−−2(R−2) P−−1(R−1|R−3)P−+1(R+1|R−1)P−+2(R+2), where Pi (Ri) is the probability of amino acid Ri occurring at the subsite i (=−6, −5, . . . , −1,+1,+2) for the sequences with a secretion-cleaved site at (−1, +1), and P− (Ri) is the corresponding probability for the sequences without any secretion-cleaved site or for those with a secretion-cleaved site located at a position other than (−1, +1). The values of the former can be derived from a positive training data set S0+ consisting of only those sequences which have a secretion-cleaved site between R−1 and R+1, and the values of the latter can be derived from a negative training data set S0− consisting of only those sequences which have no secretion-cleaved site at all or have one but its location is at any position but (−1, +1).

P+−1(R−1|R−3) is the probability of amino acid R−1 occurring at the subsite −1, given that R−3 has occurred at the subsite −3. Similarly, P++1(R+1|R−1) is the probability of amino acid R+1 occurring at the subsite +1, given that R−1 has occurred at the subsite −1. These values can be derived from a positive training data set S0+ consisting of only those sequences which have a secretion-cleaved site between R−1 and R+1 in a known manner.

P−−1(R−1|R−3) is the probability of amino acid R−1 occurring at the subsite −1, given that R−3 has occurred at the subsite −3. Similarly, P−+1(R+1|R−1) is the probability of amino acid R+1 occurring at the subsite +1, given that R−1 has occurred at the subsite −1. However, these values are derived in a known manner from a negative training data set S0+ consisting of only those sequences which have no secretion-cleaved site at all or have one but its location is at any position but (−1, +1).

The location of the cleavage site is very important because it directly correlates with an accurate prediction of the signal peptide portion 102. For example, instead of the site (−1, +1), if the cleavage site is found at (−2, −1) or (+1, +2), then the corresponding signal peptide thus derived will be one residue shorter or longer than the actual one. Therefore, for brevity hereafter only those sequences with a cleavage site (−1, +1) are called secretion-cleavable. According to the above definition, if a sequence is secretion-cleavable at (−1, +1), the value of its Ψ+ should be greater than that of Ψ−.

Accordingly, a discriminant function Δ, is given by Δ(R−6R−5R−4R−3R−2R−1R+1 R+2)=w+Ψ+(R−6R−5R−4R−3R−2R−1R+1R+2)−w−Ψ−(R−6R−5R−4R−3R−2R−1R+1R+2), where w+ and w− are the weight factors for the attribute functions derived from the positive training data set S0+ and negative training data set S0−, respectively. Typically, the weight factors are set to one (i.e., w+=w−=1). Thus, the criterion of the secretion-cleavable peptide prediction for a− given sequence can be formulated as follows. The peptide is secretion-cleavable, if its Δ>0. Otherwise, the peptide is non-secretion-cleavable. Note, that although the above method is described based on an octapeptide segment [−6, +2], a person of ordinary skill in the art will readily appreciate that any size segment [−L1, +L2] may be used.

In order to calculate the attribute function Ψ+ and Ψ− for a given sequence, we have to first find the values of Pi+(Ri) and Pi−(Ri) for (i= . . . , −2, −1, +1, +2). These values can be derived from a positive training data set S0+ and negative training data set S0−, respectively in a well known manner (e.g., the number of occurrence's is divided by the total number of samples). Preferably, the positive training data set contains only the secretion-cleavable peptides, and the negative training data set contains only the non-secretion-cleavable peptides. Preferably, redundant sequences are removed to guarantee that no pairs of homologous sequences exist in the data sets. Preferably, for the secretory proteins, the sequence of the signal peptide portion 102 and the first 30 amino acids of the mature protein portion 104 are included in the data set, while for the non-secretory proteins, the first 70 amino acids of each sequence are included. Of course a person of ordinary skill in the art will readily appreciate that any number of proteins may be included in either portion.

To compare the performance of the prediction method under equivalent conditions, the same data structure is used. By sliding the octapeptide benchmark window (or any window) along each of these sequences, the desired peptides for the training data sets S0+ and S0− are generated. The number of the non-secretion-cleavable peptides thus obtained will be much larger than that of the secretion-cleavable peptides. For example, for a secretory protein sequence which is 50 amino acids long, only one secretion-cleavable octapeptide may be generated. However, for the same sequence, (50−8) non-secretion-cleavable octapeptides can be generated. For a non-secretory protein sequence which is 70 amino acids long, (70−8+1) non-secretion-cleavable peptides may be generated, but no secretion-cleavable octapeptides may be generated. In one embodiment, 1939 secretion-cleavable octapeptides are used for data set S0+, and 179435 non-secretion-cleavable octapeptides are used for data set S0−. Increasing the length of the training peptides will gradually reduce their total number in the training data set.

The rate of correct prediction for the secretion-cleavable peptides is given by Λ+=(N+−m+)/N+, for secretion-cleavable peptides and Λ−=(N−−m−)/N−, for non-secretion-cleavable peptides. N+ represents the total number of secretion-cleavable peptides, and m+ represents the number of secretion-cleavable peptides missed in prediction. N− represents the total number of non-secretion-cleavable peptides, and m− represents the number of non-secretion-cleavable peptides incorrectly predicted as cleavable. The average rate of correct prediction for the cleavage site and hence the signal peptide concerned is given by Λ=(Λ+N++Λ−N−)/(N++N−)=1−((m++m−)/(N++N−)).

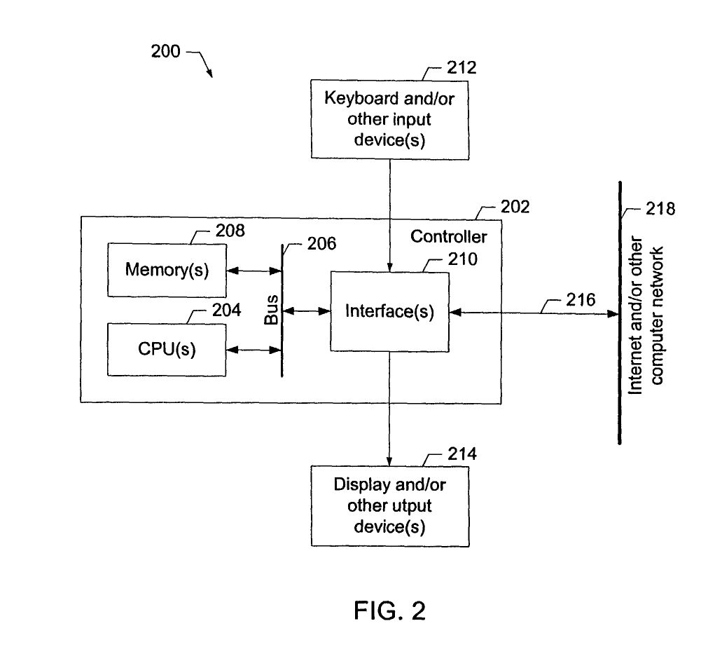

A detailed diagram of a computing device 200 capable of executing some or all of the method described herein is illustrated in FIG. 2. A controller 202 in the computing device 200 preferably includes a central processing unit 204 electrically coupled by an address/data bus 206 to a memory device 208 and an interface circuit 210. The CPU 204 may be any type of well known CPU, such as an Intel PentiumTM processor. The memory device 208 preferably includes volatile memory, such as a random-access memory (RAM), and non-volatile memory, such as a read only memory (ROM) and/or a magnetic disk. The memory device 208 stores a software program that implements all or part of the method described below. This program is executed by the CPU 204, as is well known. Some of the steps described in the method below may be performed manually or without the use of the computing device 200.

The interface circuit 210 may be implemented using any data transceiver, such as a Universal Serial Bus (USB) transceiver. One or more input devices 212 may be connected to the interface circuit 210 for entering data and commands into the controller 202. For example, the input device 212 may be a keyboard, mouse, touch screen, track pad, track ball, isopoint, and/or a voice recognition system.

An output device 214 may also be connected to the controller 202 via the interface circuit 210. Examples of output devices 214 include cathode ray tubes (CRTs), liquid crystal displays (LCDs), speakers, and/or printers. The output device 212 generates visual displays of data generated during operation of the computing device 200. The visual displays may include prompts for human operator input, run time statistics, calculated values, and/or detected data.

The computing device 200 may also exchange data with other computing devices via a connection 216 to a network 218. The connection 216 may be any type of network connection, such as an Ethernet connection. The network 218 may be any type of network, such as a local area network (LAN) and/or the Internet.

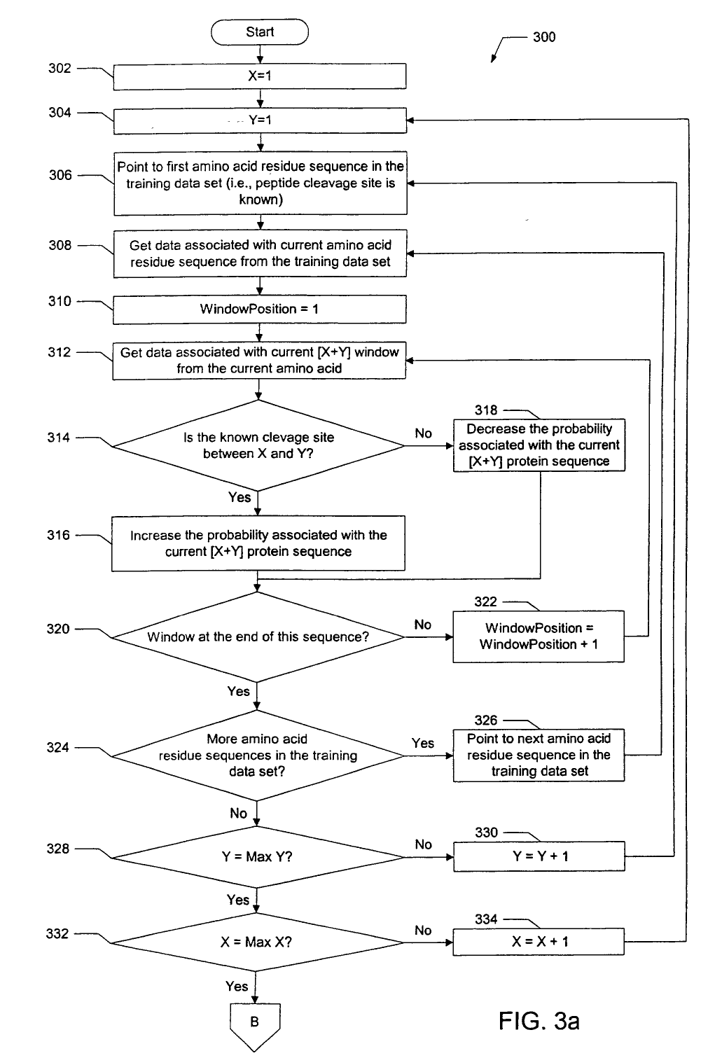

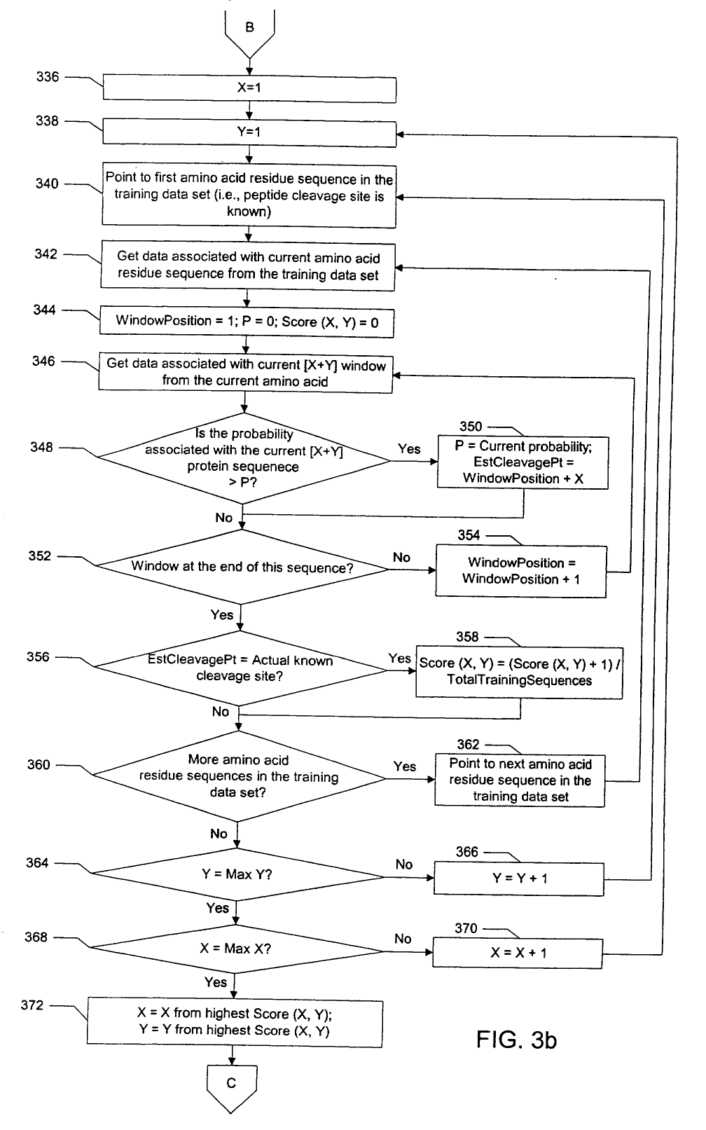

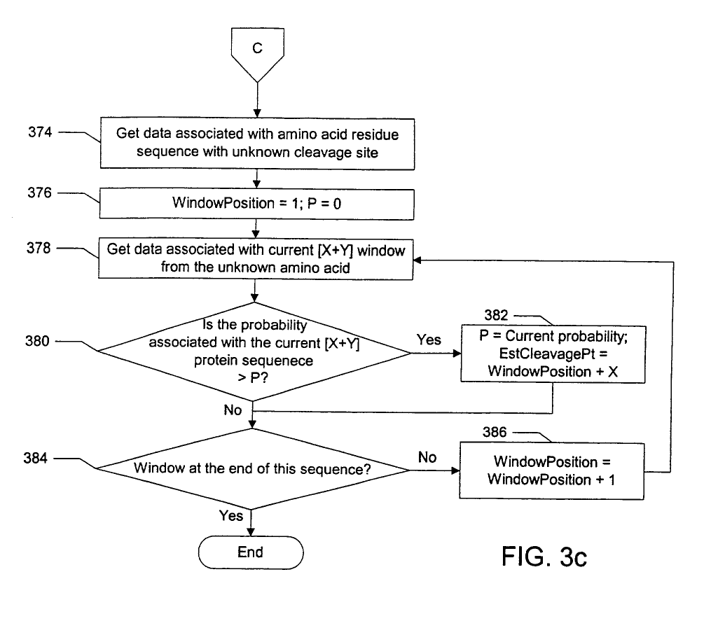

A flowchart illustrating a method 300 of predicting a signal peptide cleavage site associated with an amino acid residue sequence is illustrated in FIG. 3. The steps illustrated may be performed by the controller 202 and/or a person. Although for simplicity of discussion, these steps appear as, and will be discussed as, occurring in a particular time sequence, persons of ordinary skill in the art will readily appreciate that the method 300 can be implemented in many ways, and the disclosed steps may be executed in many temporal sequences without departing from the scope or spirit of the invention.

Generally, the method 300 determines a size (X+Y) for a residue scanning window based on a training data set. Preferably, the residue scanning window has a signal peptide portion of length X and a mature protein portion of length Y. The training data set is indicative of a plurality of amino acid residue sequences with known peptide cleavage sites. The method 300 receives a first data set representing (X+Y) amino acid residues from an amino acid residue sequence suspected of containing a signal peptide, and determines a first probability associated with the first data set based on the training data set. Subsequently, the method 300 receives a second data set representing (X+Y) amino acid residues from the same amino acid residue sequence, and determines a second probability associated with the second data set. The data set with the higher probability is chosen, thereby predicting the cleavage site to be located between X and Y. In other words, the method 300 scans the window across the amino acid residues from an amino acid residue sequence, suspected of containing a signal peptide, looking for the most likely cleavage site based on the training data.

The method 300 begins by initializing X and Y to one (steps 302-304). As described above, [X:Y] represents a residue scanning window which has a signal peptide portion of length X and a mature protein portion of length Y. Typically, a scanning window of [1:1] will not be the best predictor of the cleavage site. However, for completeness, all possible scanning windows may be tested. In an alternate embodiment, a subset of the possible scanning windows may be tested. For example, X may be initialized to six and Y may be initialized to two. In yet another alternate embodiment, a non-consecutive subset of residue positions may be used. For example, positions −3, −1, and +1 may be used. This sub-site coupling principle is discussed in detail above and below. Further, in any of the above window choices, conditional probability may be used to enhance the predicative results. For example, a Bayesian function may be incorporated into the prediction function.

After X and Y are initialized, a pointer is initialized to point to a first amino acid residue sequence in a training data set, and the data is retrieved (steps 306-308). The peptide cleavage site of this amino acid residue sequence is known. For example, data from Nielsen H. Engelbrecht, J., Brunak S., and von Heijne, G. (1997) “Identification of prokaryotic and eukaryotic signal peptides and prediction of their cleavage sites”, Protein Engineering, which is incorporated herein by reference, may be used.

Preferably, the retrieved data is scanned from “left” to “right.” Accordingly, a window position pointer is initialized to one each time a new sequence is retrieved (step 310). Of course, a person of ordinary skill in the art will readily appreciate that any other scanning order may be used without departing from the scope and spirit of the present invention. Once the window is “positioned”, the method 300 retrieves the subset of data identified by the window (step 312). If the known cleavage site is between X and Y (as determined at step 314), the method increases the probability associated with the current [X+Y] protein sequence (step 316). However, if the known cleavage site is not between X and Y, the method decreases the probability associated with the current [X+Y] protein sequence (step 318).

Once the current position of the window is tested, the method determines if the window is at the “right” end of the sequence (step 320). For example, a counter or a marker value may be used in a well known manner to detect the end of the sequence. If the window is not at the end of the sequence, the method 300 increments the window position pointer (step 322) to move the window one position to the “right.” Subsequently, the above described steps are repeated from step 312. If the window is at the end of the sequence, the method 300 determines if there are more amino acid residue sequences in the training data set (step 324). Again, a counter or a marker value may be used in a well known manner to detect the end of the training data set.

If the method 300 determines that there are more amino acid residue sequences in the training data set, it points to the next sequence in the set, and loops back to step 308 (step 326). However, if the method 300 determines that there are no more amino acid residue sequences in the training data set, it checks to see if the Y portion of the window should be increased (step 328). For example, the Y portion may be exhaustively tested, or a limited set of values, such as one—three, may be tested. If the method 300 determines that the Y portion of the window should be increased for further testing, the method 300 increments Y and loops back to step 306 (step 330).

If all desired values of Y have been tested, the method checks to see if the X portion of the window should be increased (step 332). For example, the X portion may be exhaustively tested, or a limited set of values, such as six—eighteen, may be tested. If the method 300 determines that the X portion of the window should be increased for further testing, the method 300 increments X and loops back to step 304 (step 334). If all desired values of X have been tested, the method moves on to a scoring phase of the training.

In the scoring phase of the training (FIG. 3b), the method 300 reinitializes X and Y to one (steps 336-338). As described above, [X:Y] represents a residue scanning window which has a signal peptide portion of length X and a mature protein portion of length Y. As before, a pointer is initialized to point to the first amino acid residue sequence in the training data set, and that data is retrieved (steps 340-342). As described above, the peptide cleavage site of this amino acid residue sequence is known.

If the data is scanned from “left” to “right”, a window position pointer is initialized to one each time a new sequence is retrieved (step 344). In addition, a current running probability (P) and a score variable for this selection of X and Y are initialized to zero (step 344). The score variable keeps track of how well a particular choice of X and Y for the scanning window predicts the cleavage site on the training data. Once the window is “positioned”, the method 300 retrieves that subset of the sequence data (step 346).

As the window scans the amino acid residue sequence, the method 300 determines if the probability associated with the current [X+Y] protein sequence (previously determined in steps 316-318) is greater than the current running probability (step 348). The first time through the answer will be yes, because the current running probability (P) was set to zero in step 344. If the probability associated with the current [X+Y] protein sequence is greater than the current running probability (P), the method 300 updates the current running probability (P) and temporarily determines that the estimated cleavage site is located between X and Y (i.e., the current window position plus X) (step 350). If the probability associated with the current [X+Y] protein sequence is not greater than the current running probability (P), the method 300 does not update the current running probability (i.e., looking for the maximum probability).

Once the current position of the window is tested, the method determines if the window is at the “right” end of the sequence (step 352). If the window is not at the end of the sequence, the method 300 increments the window position pointer (step 354) to move the window one position to the “right.” Subsequently, the above described steps are repeated from step 346. If the window is at the end of the sequence, the method 300 determines if the estimated cleavage site from step 350 is the actual known cleavage site (step 356). If the estimated cleavage site is correct, the method 300 increases the score for this XY combination (step 358). For example, the number of correct estimates may be divided by the total number of sequences in the training data to arrive at a percentage of accuracy.

Subsequently, the method 300 determines if there are more amino acid residue sequences in the training data set (step 324). If the method 300 determines that there are more amino acid residue sequences in the training data set, it points to the next sequence in the set, and loops back to step 342 (step 362). However, if the method 300 determines that there are no more amino acid residue sequences in the training data set, it checks to see if the Y portion of the window should be increased (step 328). If the method 300 determines that the Y portion of the window should be increased for further testing, the method 300 increments Y and loops back to step 340 (step 366).

If all desired values of Y have been tested, the method checks to see if the X portion of the window should be increased (step 332). If the method 300 determines that the X portion of the window should be increased for further testing, the method 300 increments X and loops back to step 338 (step 370). If all desired values of X have been tested, the method determines the desired value of X and Y for the scanning of residue sequences with unknown cleavage sites (step 372). This determination may be made by taking the value of X and Y which are associated with the largest score from step 358.

Once the training is completed and a desired residue scanning window [X:Y] is determined, the method 300 is ready to estimate the cleavage site of amino acid residue sequences with unknown cleavage sites. Accordingly, the method 300 retrieves data associated with an amino acid residue sequence having an unknown cleavage site (step 374). In keeping with the above, the data is scanned from “left” to “right”, therefore, a window position pointer is initialized to one (step 376). In addition, a current running probability (P) is preferably initialized to zero (step 376). Once the window is “positioned”, the method 300 retrieves that subset of the sequence data (step 378).

As the window scans the amino acid residue sequence, the method 300 determines if the probability associated with the current [X+Y] protein sequence (previously determined in steps 316-318) is greater than the current running probability (step 380). The first time through the answer will be yes, because the current running probability (P) was set to zero in step 376. If the probability associated with the current [X+Y] protein sequence is greater than the current running probability (P), the method 300 updates the current running probability (P) and temporarily determines that the estimated cleavage site is located between X and Y (step 382). If the probability associated with the current [X+Y] protein sequence is not greater than the current running probability (P), the method 300 does not update the current running probability (i.e., looking for the maximum probability).

Once the current position of the window is tested, the method determines if the window is at the “right” end of the sequence (step 384). If the window is not at the end of the sequence, the method 300 increments the window position pointer (step 386) to move the window one position to the “right.” Subsequently, the above described steps are repeated from step 378. If the window is at the end of the sequence, the method 300 may end. When the method ends, the estimated cleavage site is available in the variable “EstCleavgePt” as determined by step 382.

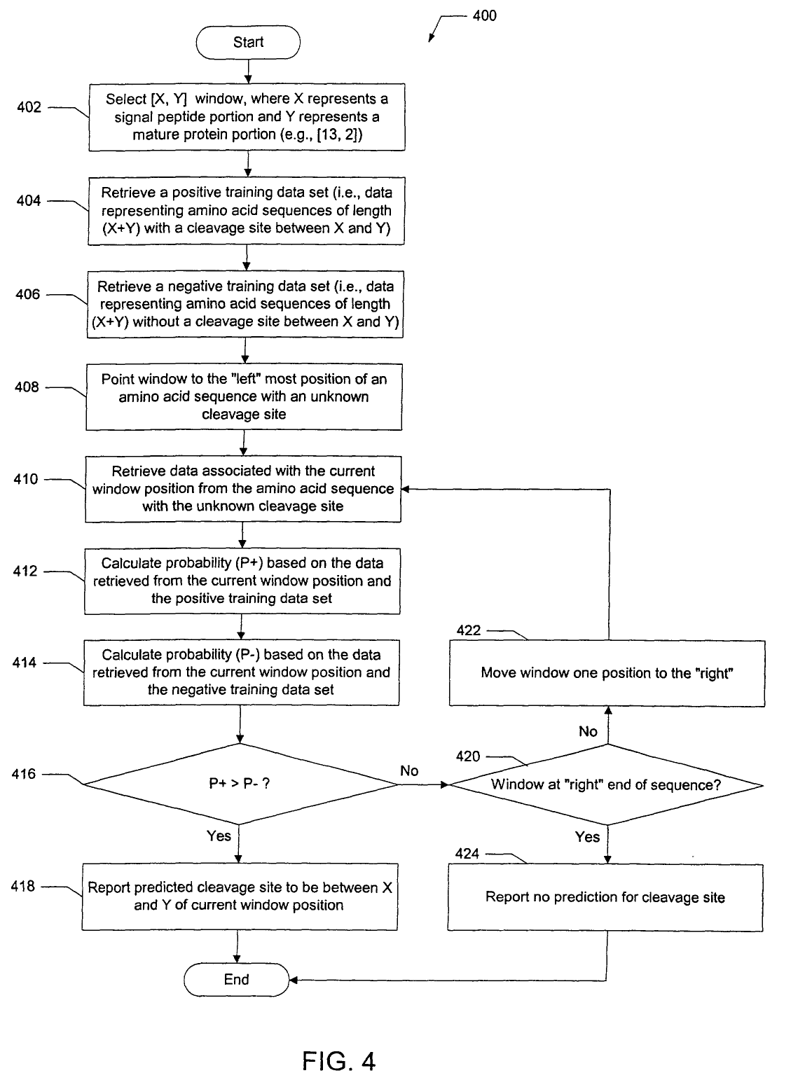

A flowchart illustrating another method 400 of predicting a signal peptide cleavage site associated with an amino acid residue sequence is illustrated in FIG. 4. The steps illustrated may be performed by the controller 202 and/or a person. Although for simplicity of discussion, these steps appear as, and will be discussed as, occurring in a particular time sequence, persons of ordinary skill in the art will readily appreciate that the method 400 can be implemented in many ways, and the disclosed steps may be executed in many temporal sequences without departing from the scope or spirit of the invention.

Generally, the method 400 calculates and compares two probabilities. The first probability (P+) is based on data retrieved from a scanning window and a positive training data set. The second probability (P−) is based on the same scanning window data, but a negative training data set is used. When P+ is greater than P−, the cleavage site is predicted to be within the current window between a signal peptide portion of length X and a mature protein portion of length Y. The two probabilities may be based on independent elements (i.e., no coupling among sub-sites), or the two probabilities may be based on coupled elements. For example, positions −3, −1, and +1 may be used as described above. In addition, the probabilities for a query peptide sequence may be computed as conditional probabilities according to Markov chain theory.

The method 400 begins by selecting an [X, Y] window, where X represents a signal peptide portion and Y represents a mature protein portion (step 402). For example a window [13,2] having a signal peptide portion of length 13 and a mature protein portion of length 2 may be used. Subsequently, the method 400 retrieves a positive training data set (step 404) and a negative trains data set (step 406). Each member of the positive training data set preferably represents an amino acid sequences of length (X+Y) with a cleavage site between X and Y. Each member of the negative training data set preferably represents an amino acid sequences of length (X+Y) with no cleavage site between X and Y. A pointer is then initializes to point to the “left” side of an amino acid sequence containing an unknown cleavage site (step 408).

The method 400 then enters a scanning loop to determine the cleavage site. Data associated with the amino acid sequence is retrieved from the current position of the scanning window (step 410). This data is then used with the positive training data set to calculate a first probability P+ (step 412), and with the negative training data set to calculate a second probability P− (step 414). If the first probability P+ is greater than the second probability P− (step 416), the method 400 reports the predicted cleavage site to be between X and Y of the current window position (step 418) and ends.

If the first probability P+ is not greater than the second probability P− (step 416), the method 400 checks if the entire amino acid sequence has been scanned (step 420). If the entire amino acid sequence has not been scanned, the method 400 move the scanning window one position to the “right” (step 422) and repeats the process from step 410. If the entire amino acid sequence has been scanned without locating a cleavage site, the method 400 reports that no cleavage site prediction was made (step 424) and ends.

Once an estimated cleavage site of a peptide with an unknown cleavage site is determined, the program is ready to prepare a chimeric polynucleotide encoding for the estimated mature protein. The computing device 200 may exchange data with a program which translates amino acid sequences into a corresponding polynucleotide sequence which encodes for the original amino acid sequence, and an automated polynucleotide synthesizer which can be programmed to produce polynucleotides of variable length. Once the method 400 has estimated the cleavage site of an amino acid sequence having an unknown cleavage site, the program may then translate the amino acid sequence examined by the method into a polynucleotide sequence which encodes for the protein. This polynucleotide sequence is transferred to the automated polynucleotide synthesizer, and the synthesizer then prepares a polynucleotide encoding for an expression control sequence fused to all or a portion of the amino acid sequence examined by the program. For example, after estimation of the cleavage site within an amino acid sequence with unknown cleavage sites, data may be transmitted to the sequencer for preparation of a chimeric polynucleotide encoding for an expression control sequence fused with the estimated polynucleotide sequence encoding for the mature protein. After the chimeric polynucleotide is obtained from the sequencer, the polynucleotide sequence may then be transfected into a host cell, the sequence expressed, and the expressed recombinant polypeptide purified from the host cell or the growth media of the cell.

The computing device 200 may also exchange data with an automated peptide synthesizer, allowing the program to directly prepare a synthetic polypeptide comprising the estimated mature sequence determined by the method 400. Alternatively, the automated peptide synthesizer may be programmed to prepare a synthetic amino acid sequence comprising a signal peptide fused N-terminal to the estimated mature protein, with the provision that the signal peptide does not include the original peptide sequence fused to and immediately upstream of the predicted mature protein portion of the sequence. The resulting synthetic peptide may then be tested for activity or folding in vitro or in vivo.

This system facilitates recombinant protein production of any mature protein (and production of synthetic polynucleotides encoding such a mature protein) by virtue of the production of a signal peptide cleavage site as described herein.

A variety of expression vector/host systems may be utilized to contain and express a particular coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein productions include but are not limited VERO cells, HeLa cells, Chinese hamster ovary (CHO) cell lines, COS cells (such as COS-7), W138, BHK, HepG2, 3T3, RIN, MDCK, A549, PC12, K562 and 293 cells. Recombinant protein expression in these systems is described in further detail in this example.

The DNA sequence encoding the mature form of a protein is amplified (e.g., by PCR) and cloned into an appropriate vector for example, pGEX-3X (Pharmacia, Piscataway, N.J.). The pGEX vector is designed to produce a fusion protein comprising glutathione-S-transferase (GST), encoded by the vector, and a protein encoded by a DNA fragment inserted into the vector's cloning site. The primers for the PCR may be generated to include for example, an appropriate restriction endonuclease cleavage site to facilitate cloning.

Treatment of the recombinant fusion protein with thrombin or factor Xa (Pharmacia, Piscataway, N.J.) is expected to cleave the fusion protein, releasing the recombinant protein from the GST portion. The pGEX-3X/polynucleotide construct is transformed into E. coli XL-1 Blue cells (Stratagene, La Jolla Calif.), and individual transformants are isolated and grown. Plasmid DNA from individual transformants can then be purified and partially sequenced using an automated sequencer to confirm the presence of the desired gene insert in the proper orientation.

Using DNA sequences that encode a mature protein methods of the present example are used for the modification of cells to permit introduction of or increase expression of such a protein. The cells can be modified (heterologous promoter is inserted in such a manner that it is operably linked to, by homologous recombination) to provide increased protein expression by replacing, in whole or in part the naturally occurring protein promoter with all or part of a heterologous promoter so that the cells express the protein at higher levels. The heterologous promoter is inserted in such a manner that it is operably linked to protein-encoding sequences. (e.g., PCT International Publication No. WO96/12650; PCT International Publication No. WO 92/20808 and PCT International Publication No. WO 91/09955). It is contemplated that, in addition to the heterologous promoter DNA, amplifiable marker DNA (e.g., ada, dhfr and the multifunctional CAD gene which encodes carbamyl phosphate synthase, aspartate transcarbamylase and dihydroorotase) and/or intron DNA may be inserted along with the heterologous promoter DNA. If linked to the protein coding sequence, amplification of the marker DNA by standard selection methods results in co-amplification of the protein coding sequences in the cells.

Alternatively, the DNA sequence encoding the predicted mature protein may be cloned into a plasmid containing a desired promoter and, optionally, a heterologous leader sequence [see, e.g., Better et al., Science, 240:1041-43 (1988)]. The sequence of this construct may be confirmed by automated sequencing. The plasmid is then transformed into an appropriate bacterial strain using standard procedures employing CaC12 incubation and heat shock treatment of the bacteria (Sambrook et al., supra).

E. coli is a preferred prokaryotic host. For example, E. coli strain RR1 is particularly useful. Other microbial strains which may be used include E. coli strains such as E. coli LE392, E. coli B, and E. coli X 1776 (ATCC No. 31537). The aforementioned strains, as well as E. coli W3 110 (F-, lambda-, prototrophic, ATCC No. 273325), bacilli such as Bacillus subtilis, or other enterobacteriaceae such as Salmonella typhimurium or Serratia marcescens, and various Pseudomonas species may be used. These examples are, of course, intended to be illustrative rather than limiting.

The transformed bacteria are grown in any of a number of suitable media, for example LB, and the expression of the recombinant polypeptide induced by adding IPTG to the media or switching incubation to a higher temperature. After culturing the bacteria for a further period of between 2 and 24 hours, the cells are collected by centrifugation and washed to remove residual media. If present, the leader sequence will effect secretion of the mature protein and be cleaved during secretion. The bacterial cells are then lysed, for example, by disruption in a cell homogenizer and centrifuged to separate the dense inclusion bodies and cell membranes from the soluble cell components. This centrifugation can be performed under conditions whereby the dense inclusion bodies are selectively enriched by incorporation of sugars such as sucrose into the buffer and centrifugation at a selective speed.

If the recombinant protein is expressed in the inclusion bodies, as is the case in many instances, these can be washed in any of several solutions to remove some of the contaminating host proteins, then solubilized in solutions containing high concentrations of urea (e.g. 8M) or chaotropic agents such as guanidine hydrochloride in the presence of reducing agents such as—mercaptoethanol or DTT (dithiothreitol).

Once the mature protein is secreted into the media, the protein can then be purified and separated from the components of the media by chromatography on any of several supports including ion exchange resins, gel permeation resins or on a variety of affinity columns.

Alternatively, protein may be recombinantly expressed in yeast using a commercially available expression system, e.g., the Pichia Expression System (Invitrogen, San Diego, Calif.), following the manufacturer's instructions. This system relies on the pre-pro-alpha sequence to direct secretion of the mature polypeptide. In this system but transcription of the polynucleotide insert is driven by the alcohol oxidase (AOX1) promoter upon induction by methanol. Other systems are known or can be engineered comprising alternative promoters and leader sequences, e.g., Kurjan and Herskowitz, Cell, 30:933-943 (1982); Rose and Broach, Meth. Enz. 185:234-279, D. Goeddel, ed., Academic Press, Inc., San Diego, Calif. (1990); Price et al., Gene, 55:287 (1987); Bitter et. al., Proc. Natl. Acad. Sci. USA, 81:5330-5334 (1984). The secreted recombinant protein is purified from the yeast growth medium using standard techniques.

Alternatively, the cDNA may be cloned into the baculovirus expression vector pVL1393 (PharMingen, San Diego, Calif.). This vector is then used according to the manufacturer's directions (PharMingen) to infect Spodoptera frugiperda cells in sF9 protein-free media and to produce recombinant protein. The protein is purified and concentrated from the media using a heparin-Sepharose column (Pharmacia, Piscataway, N.J.) and sequential molecular sizing columns (Amicon, Beverly, Mass.), and resuspended in PBS. SDS-PAGE analysis is then used to show size and purity of the protein extract.

Insect systems for protein expression also are well known to those of skill in the art. In one such system, Autographa californica nuclear polyhedrosis virus (AcNPV) is used as a vector to express foreign genes in Spodoptera frugiperda cells or in Trichoplusia larvae. The polynucleotide is cloned into a nonessential region of the virus, such as the polyhedrin gene, and placed under control of the polyhedrin promoter. Successful insertion of sequence will render the polyhedrin gene inactive and produce recombinant virus lacking coat protein coat. The recombinant viruses are then used to infect S. frugiperda cells or Trichoplusia larvae in which the recombinant protein is expressed (Smith et al. (1983) J Virol 46:584; Engelhard EK et al (1994) Proc Nat Acad Sci 91:3224-7).

Mammalian host systems for the expression of the recombinant protein also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, W138, and the like have specific cellular machinery and characteristic mechanisms for such post-translational activities and may be chosen to ensure the correct modification and processing of the introduced, foreign protein.

It is preferable that the transformed cells are used for long-term, high-yield protein production and as such stable expression is desirable. Once such cells are transformed with vectors that contain selectable markers along with the desired expression cassette for encoding a given protein, the cells may be allowed to grow for 1-2 days in an enriched media before they are switched to selective media. The selectable marker is designed to confer resistance to selection and its presence allows growth and recovery of cells which successfully express the introduced sequences. Resistant clumps of stably transformed cells can be proliferated using tissue culture techniques appropriate to the cell.

A number of selection systems may be used to recover the cells that have been transformed for recombinant protein production. Such selection systems including, but not limited to, HSV thymidine kinase, hypoxanthine-guanine phosphoribosyltransferase and adenine phosphoribosyltransferase genes, in tk-, hgprt- or aprt- cells, respectively. Also, anti-metabolite resistance can be used as the basis of selection for dhfr, that confers resistance to methotrexate; gpt, that confers resistance to mycophenolic acid; neo, that confers resistance to the aminoglycoside G418; als which confers resistance to chlorsulfuron; and hygro, that confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins,—glucuronidase and its substrate, GUS, and luciferase and its substrate, luciferin.

In summary, persons of ordinary skill in the art will readily appreciate that a method and apparatus for predicting a signal peptide cleavage site associated with an amino acid residue sequence has been provided. However, the foregoing description has been presented for the purposes of illustration and description only. It is not intended to be exhaustive or to limit the invention to the precise form disclosed. Many modifications and variations are possible in light of the above teachings. It is intended that the scope of the invention be limited not by this detailed description, but rather by the claims appended hereto.

What is claimed is:

1. A method for predicting a signal peptide cleavage site associated with an amino acid sequence, the method comprising the steps of:
determining a size (X+Y) for a scanning window based on a training data set, the scanning window having a signal peptide portion of length X and a mature protein portion of length Y, the training data set being indicative of a plurality of amino acid sequences with known peptide cleavage sites;
receiving a first data set representing (X+Y) amino acids from an amino acid sequence suspected of containing a signal peptide;
determining a first probability associated with the first data set based on the training data set;
receiving a second data set representing (X+Y) amino acids from the amino acid sequence suspected of containing a signal peptide;
determining a second probability associated with the second data set based on the training data set; and
selecting the first data set if the first probability is greater than the second probability.

2. A method as defined in claim 1, wherein the step of determining a first probability associated with the first data set based on the training data set includes the step of determining a conditional probability.

3. A method as defined in claim 2, wherein the step of determining a conditional probability includes the step of calculating values associated with a Markov chain.

4. A method as defined in claim 2, wherein the step of determining a conditional probability includes the step of determining a conditional probability associated with subsites −3, −1, and +1.

5. A method as defined in claim 2, wherein the step of determining a conditional probability includes the step of determining a conditional probability associated with subsites −2, −1, and +1.

6. A method as defined in claim 2, wherein the step of determining a conditional probability includes the step of determining a conditional probability associated with subsites −3, −2,−1, and +1.

7. A method as defined in claim 1, wherein the step of determining a size (X+Y) for a scanning window includes the step of determining the size (X+Y) to be between five residues and thirty residues.

8. A method as defined in claim 1, wherein the step of determining a size (X+Y) for a scanning window includes the step of determining the size (X+Y) to be between seven residues and twenty-one residues.

9. A method as defined in claim 1, wherein the step of determining a size (X+Y) for a scanning window includes the step of determining the size (X+Y) to be fifteen residues.

10. A method as defined in claim 1, wherein the step of determining a size (X+Y) for a scanning window includes the step of determining a signal peptide portion of length X to be between five and twenty-five residues.

11. A method as defined in claim 1, wherein the step of determining a size (X+Y) for a scanning window includes the step of determining a signal peptide portion of length X to be between ten and sixteen residues.

12. A method as defined in claim 1, wherein the step of determining a size (X+Y) for a scanning window includes the step of determining a signal peptide portion of length X to be thirteen residues.

13. A method as defined in claim 12, wherein the step of determining a size (X+Y) for a scanning window includes the step of determining a mature protein portion of length Y to be two residues.

14. A method as defined in claim 1, wherein the step of determining a size (X+Y) for a scanning window includes the step of determining a mature protein portion of length Y to be between one residue and five residues.

15. A method as defined in claim 1, wherein the step of determining a size (X+Y) for a scanning window includes the step of determining a mature protein portion of length Y to be two residues.

16. A method as defined in claim 1, wherein the step of receiving a first data set representing (X+Y) amino acids from an amino acid sequence includes the step of receiving a first data set representing (X+Y) consecutive amino acids from the amino acid sequence.

17. A method as defined in claim 16, wherein the step of receiving a second data set representing (X+Y) amino acids from an amino acid sequence includes the step of receiving a second data set representing (X+Y) consecutive amino acids from the amino acid sequence.

18. A method as defined in claim 17, wherein the first data set differs from the second data set by only one window position.

19. A method as defined in claim 1, wherein the step determining a first probability associated with the first data set based on the training data set includes the step of retrieving a previously stored probability associated with the training data set.

20. A method as defined in claim 1, further comprising a step of preparing a chimeric nucleotide sequence comprising an expression control nucleotide sequence fused in frame with a nucleotide sequence encoding the mature protein portion of the amino acid sequence.

21. A method as defined by claim 20, further comprising the steps of:
transforming or transfecting a host cell with the chimeric nucleotide sequence; and
growing the host cell under conditions to permit expression of the polypeptide encoded by the chimeric nucleotide sequence.

22. A method as defined by claim 21, further comprising the step of purifying the polypeptide from the host cell or the growth media of the cell.

23. A method as defined by claim 21, wherein the expression control sequence includes a heterologous signal peptide sequence fused in frame with the nucleotide sequence encoding the mature protein.

24. A method as defined by claim 21, wherein the host cell is a eukaryotic cell that recognizes and cleaves the heterologous signal peptide and secretes a polypeptide encoded by the chimeric nucleotide sequence and lacking the signal peptide.

25. A method as defined in claim 1, further comprising the step of preparing a synthetic polypeptide comprising the mature protein sequence and lacking the signal peptide.

26. A method as defined in claim 25, wherein the synthetic peptide consists of the mature protein sequence.

27. A method as defined in claim 25, wherein the synthetic peptide comprises a tag amino acid sequence fused to the amino terminus of the mature protein sequence.

28. An apparatus for predicting a signal peptide cleavage site associated with an amino acid sequence, the apparatus comprising:
a memory device storing a software program; and
a central processing unit operatively coupled to the memory device, the central processing unit executing the software program;
the software program determining a size (X+Y) for a scanning window based on a training data set, the scanning window having a signal peptide portion of length X and a mature protein portion of length Y, the training data set being indicative of a plurality of amino acid sequences with known peptide cleavage sites;
the software program receiving a first data set representing (X+Y) amino acids from an amino acid sequence suspected of containing a signal peptide;
the software program determining a first probability associated with the first data set based on the training data set;
the software program receiving a second data set representing (X+Y) amino acids from the amino acid sequence suspected of containing a signal peptide;
the software program determining a second probability associated with the second data set based on the training data set; and
the software program selecting the first data set if the first probability is greater than the second probability.

29. An apparatus as defined in claim 28, wherein the software program determines the first probability associated with the first data set based on the training data set by determining a conditional probability.

30. An apparatus as defined in claim 29, wherein the software program determines the first probability associated with the first data set based on the training data set by determining a Markov chain.

31. An apparatus as defined in claim 29, wherein the conditional probability is based on subsites −3, −1, and +1.

32. An apparatus as defined in claim 29, wherein the conditional probability is based on subsites −2, −1, and +1.

33. An apparatus as defined in claim 29, wherein the conditional probability is based on subsites −3, −2, −1, and +1.

34. An apparatus as defined in claim 28, wherein the software program determines the size (X+Y) to be between five residues and thirty residues.

35. An apparatus as defined in claim 28, wherein the software program determines the size (X+Y) to be fifteen residues.

36. An apparatus as defined in claim 28, wherein the software program determines the signal peptide portion of length X to be between five and twenty-five residues.

37. An apparatus as defined in claim 28, wherein the software program determines the signal peptide portion of length X to be thirteen residues.

38. An apparatus as defined in claim 37, wherein the software program determines the mature protein portion of length Y to be to be two residues.

39. An apparatus as defined in claim 28, wherein the software program determines the mature protein portion of length Y to be between one residue and five residues.

40. An apparatus as defined in claim 28, wherein the software program determines the mature protein portion of length Y to be two residues.

41. An apparatus as defined in claim 28, wherein the software program receives a first data set representing (X+Y) consecutive amino acids from an amino acid sequence.

42. An apparatus as defined in claim 28, wherein the software program retrieves a previously stored probability associated with the training data set.

43. A computer readable medium storing a software program, the software program representing the steps of:
determining a size (X+Y) for a scanning window based on a training data set, the scanning window having a signal peptide portion of length X and a mature protein portion of length Y, the training data set being indicative of a plurality of amino acid sequences with known peptide cleavage sites;
receiving a first data set representing (X+Y) amino acids from an amino acid sequence suspected of containing a signal peptide;
determining a first probability associated with the first data set based on the training data set;
receiving a second data set representing (X+Y) amino acids from the amino acid sequence suspected of containing a signal peptide;
determining a second probability associated with the second data set based on the training data set; and
selecting the first data set if the first probability is greater than the second probability.

44. A computer readable medium as defined in claim 43, wherein the step of determining a first probability associated with the first data set based on the training data set includes the step of determining a conditional probability.

45. A computer readable medium as defined in claim 44, wherein the step of determining a first probability associated with the first data set based on the training data set includes the step of determining a Markov chain.

46. A computer readable medium as defined in claim 44, wherein the conditional probability is based on subsites −3, −1, and +1.

47. A computer readable medium as defined in claim 44, wherein the conditional probability is based on subsites −2, −1, and +1.

48. A computer readable medium as defined in claim 44, wherein the conditional probability is based on subsites −3, −2, −1, and +1.

49. A method of using a computer to predict a signal peptide cleavage site, the method comprising the steps of:
programming the computer to employ a scanning window, the scanning window representing a signal peptide portion and a mature protein portion;
entering data indicative of an amino acid sequence with an unknown cleavage site; and
receiving an output from the computer reporting a predicted cleavage site for the amino acid sequence.

50. A method as defined in claim 49, further comprising the step of programming the computer to determine a conditional probability.

51. A method as defined in claim 50, further comprising the step of programming the computer to determine a Markov chain.

52. A method as defined in claim 50, wherein the conditional probability is based on subsites −3, −1, and +1.

53. A method as defined in claim 50, wherein the conditional probability is based on subsites −2, −1, and +1.

54. A method as defined in claim 50, wherein the conditional probability is based on subsites −3, −2, −1, and +1.

55. A method as defined in claim 49, wherein the step of programming the computer to employ a scanning window includes the step of programming the computer to employ a scanning window representing a signal peptide portion with a length of thirteen residues and a mature protein portion with a length of two residues.

56. A method as defined in claim 49, further comprising a step of preparing a chimeric nucleotide sequence comprising an expression control nucleotide sequence fused in frame with a nucleotide sequence encoding the mature protein portion of the amino acid sequence.

57. A method as defined by claim 56, further comprising the steps of:
transforming or transfecting a host cell with the chimeric nucleotide sequence; and
growing the host cell under conditions to permit expression of the polypeptide encoded by the chimeric nucleotide sequence.

58. A method as defined by claim 57, further comprising the step of purifying the polypeptide from the host cell or the growth media of the cell.

59. A method as defined by claim 57, wherein the expression control sequence includes a heterologous signal peptide sequence fused in frame with the nucleotide sequence encoding the mature protein.

60. A method as defined by claim 57, wherein the host cell is a eukaryotic cell that recognizes and cleaves the heterologous signal peptide and secretes a polypeptide encoded by the chimeric nucleotide sequence and lacking the signal peptide.

61. A method as defined in claim 49, further comprising the step of preparing a synthetic polypeptide comprising the mature protein sequence.

62. A method as defined in claim 49, further comprising the step of preparing a synthetic polypeptide comprising a signal peptide fused with the mature protein sequence.
